(19) United States
(12) Patent Application Publication
(10) Pub. No.: US 2022/0040002 A1
(43) Pub. Date: Feb. 10, 2022

(54) SKI GOGGLES WITH DETACHABLE LENS

(71) Applicant: Active Brands AS, Oslo (NO)

(72) Inventors: Ståle Norman MØLLER, Trysil (NO); Dan Martin Fjørde SEVALDSON, Oslo (NO)

(21) Appl. No.: 17/275,655

(22) PCT Filed: Sep. 12, 2019
(86) PCT No.: PCT/EP2019/074367

§ 371 Date: Mar. 11, 2021

(30) Foreign Application Priority Data
Sep. 14, 2018 (NO) 20181201

(51) Int. Cl. A61F 9/02; G02B 7/14; G02B 7/02

(52) U.S. Cl. CPC A61F 9/025; A63C 11/00; G02B 7/026; G02B 7/14

(57) ABSTRACT

A ski goggles with detachable lens comprises a frame constituted by a lens bedding part (1) for receiving lens (3) and being attached to a soft polymer frame part (2), so as to work as one piece. A lens panel is defined and laminated between the outer lens and the inner lens. Magnets are provided in the lens bedding (1) for automatic and correct location of the lens (3). A tab (4) with polymer spring is provided centrally on the lens panel, which is received by a locking lever (5) located on the lens bedding part (1) for locking the lens. The magnets are adapted to locate the lens (3) on the lens bedding part (1) such that when the locking lever (5) is released, the lens protrude out of the frame keeping a reasonable gap from the frame.

### TECHNICAL FIELD

The present invention in general, relates to ski goggles with detachable lens systems.

Particularly, the present invention relates to ski goggles with a detachable lens system and a lens exchange mechanism wherein the lens can be easily exchanged without the need to touch the lens surface and thereby avoid finger marks on the lens.

### BACKGROUND ART

Skiing is an important sports activity that is gaining popularity around the globe. This sport requires specific and specialized sporting gear, without which it is not possible to take part in this activity. Ski goggles are one of the most important skiing accessories. The main purpose of the skiing goggles is to provide a clear view of the skiing track to the wearer in very different types of weather conditions. They must be comfortable for the wearer to wear for long periods of time and must have provision for ventilation to prevent fogging.

As the skier travels at elevated speeds during skiing, it is of utmost importance to ensure that he gets a clear view of the skiing track in front of him under all conditions of light. It is well known that the undulated snow of the track produces different reflections of the light falling on the snow, thereby creating various light conditions. The ski goggles must therefore be suitable for all these light conditions. This gives rise to interchangeable lenses for ski goggles.

Ski goggles with detachable and interchangeable lenses are well known. Different lenses are provided to take care of the various light conditions mentioned above. The advantages with a detachable lens system is that it is possible to replace lenses with different absorbance of light in order to obtain optimal contrast under different snow/light conditions.

The skier has to change the lens to suit his eyes according to the light condition existing on the track.

Granted U.S. Pat. No. 3,896,496 discloses an interchangeable lens ski goggle especially suitable for active sports such as skiing and motorcycling. An important feature of the goggles is the special structure of such interchangeable lens means which provide for both ease in interchangeability of lenses while at the same time providing a stronger support around the periphery of the lens so that the lens is supported in a manner to provide the protection needed. But this invention does not deal with the problem of leaving finger marks on the lens surface while changing the lens, which the present invention addresses.

US 2013/0104300A1 describes a device for detachably fixing a lens on goggles, making use of permanent magnets and fastening means on each side of the lens.

Ski goggles of the prior art are provided with lens exchange mechanisms which are not easy to operate, particularly as the skier has gloves/mitten on his hands, through which he must change the lens. Also, the changing of lenses usually leaves finger marks on the lenses.

The operation of changing the ski goggles lenses in the prior art takes appreciable amount of time too.

Accordingly, there is need for ski goggles with interchangeable detachable lenses which offer quick and effortless changing of lenses even with gloves/mitten on, and which do not leave finger marks on the lens after changing them.

The present invention seeks to meet these requirements.

### OBJECTS OF THE INVENTION

### Summary of Invention

Accordingly, the present invention provides a ski goggles with detachable lens comprising a frame constituted by a lens bedding part for receiving lens and being attached to a soft polymer frame part so as to work as one piece. A lens panel is defined and laminated between the outer lens and the inner lens, wherein magnets are provided in the lens bedding part end the lens panel for automatic and correct location of the lens. A tab with central spring is provided on the lens panel, where the central spring of the tab is received by a notch in a locking lever located on the lens bedding part, where rotation of the locking lever locks the lens to the lend bedding part, said magnets are adapted to locate the lens on the lens bedding part such that once the locking lever is released and the engagement between the notch and the central spring is released, the lens protrudes out of the lens bedding part keeping a gap from the lens bedding part.

Further preferred embodiments are described in the dependent claims.

The present invention also provides a method of locking a lens in place in the ski goggles with detachable lens as hereinbefore described, characterized by the steps of:
inserting the lens into the frame with the lens protruding out from the frame leaving a gap, when a central spring of a tab enters a notch in the locking lever;
rotating the locking lever downwards for locking the lens, when the central spring in the tab moves up; and
rotating the locking lever fully to its bottom most position, when the lens moves inwards, whereby the central spring in the tab releases its tension and locks the lens in its place.

The present invention also provides a method of unlocking a lens which is in place in the ski goggles with detachable lens as hereinbefore described characterized by:
rotating the locking lever upwards to release the lens, when the central spring of the tab 4 is pushed up; and
unlocking the lens and pushing it out of the frame to protrude out leaving a gap from the frame, when the locking lever 5 is in the fully open position, whereby the lens can easily be changed without touching its surface.

Further preferred embodiments are described in the dependent claims.

### BRIEF DESCRIPTION OF DRAWINGS

Having described the main features of the invention above, a more detailed and non-limiting description of a preferred embodiment will be given in the following with reference to the drawings.

FIG. 1 is a top view of the lens bedding, where the lens is attached.

FIG. 2 is a top view of the soft polymer frame part (TPU) where the foam towards the face is attached.

FIG. 3 is a top view of the frame when the two parts shown in FIGS. 1 and 2 are molded together.

FIG. 4 is a side view of an interchangeable lens of the ski goggles according to the present invention.

FIG. 5 is a cross section view of the lens and lens panel, made of injection molded nylon, containing magnets for locating the lens and the tab in the center of the lens panel having polymer spring which is received by the locking lever with notch for the purpose of receiving the lens and locking it in place.

FIG. 6 is a perspective view of the goggles frame showing the lens bedding, which is part of the frame, containing magnets for locating the lens, as well as a housing for the locking lever.

FIG. 7 is a perspective view of the frame shown in FIG. 6 with the lens fitted.

FIG. 8 is a cross section view of the lens and lens panel shown in FIG. 5 showing how the tab on lens panel is received by the locking lever.

FIG. 9 is a view of the lens moved into the frame when the locking lever is rotated, providing a secure attachment of the lens with the frame.

FIG. 10 is a cross section view of the locking lever in the locked condition.

FIG. 11 is a view of how the lens is inserted into the frame.

FIG. 12 is a view of upward movement of the center spring in the tab when rotating the locking lever downwards for locking the lens.

FIG. 13 is a view of the locking lever fully rotated downwards locking the lens in place.

FIG. 14 is a view of upward rotation of the locking lever to release the lens.

FIG. 15 is a view of the center spring in the tab being pushed up while the locking lever is rotating upwards for releasing the lens.

FIG. 16 is a view of the locking lever in the fully open position to release the lens.

### DETAILED DESCRIPTION OF THE INVENTION

The following describes a preferred embodiment of the present invention which is purely exemplary for the sake of understanding the invention and non-limiting.

It should be understood that the number of components may be different from what is shown in the drawings without deviating from the principle of the invention.

In all the figures, like reference numerals represent like features.

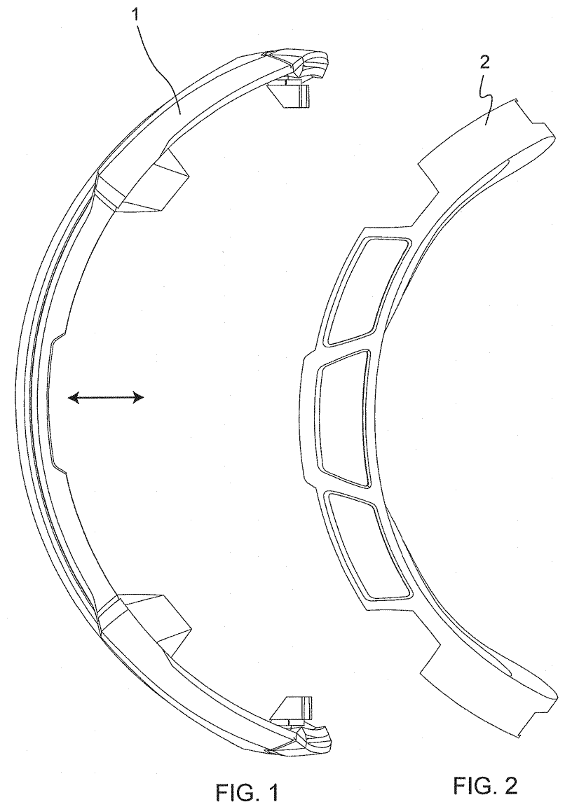

Referring to FIGS. 1, 2 and 3, the ski goggles according to the present invention has a lens bedding 1 made of carbon fiber reinforced nylon. This is where the lens 3 is attached. The bedding is attached to a soft polymer frame part 2, made of thermoplastic polyurethane (hereinafter referred to as TPU) where the foam towards the face is attached.

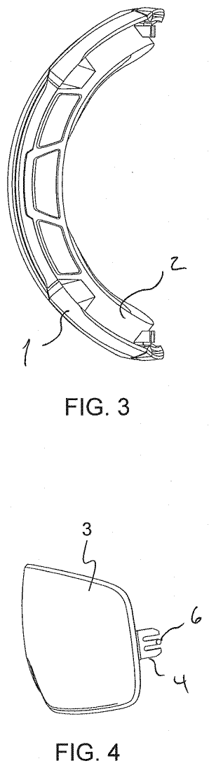

The lens bedding 1 is made of a rigid material to make sure there is a rigid and precise interface for the lens in order to maintain the optical properties of the lens. Bending the lens to adapt to the face affects the optical properties. The TPU 2, which is a softer material, adapts to the contours of the face. The two parts are molded together to work as one piece. The combination of the bedding 1 and TPU 2 is shown in FIG. 3.

FIG. 4 is a side view of an interchangeable lens 3 of the ski goggles fitted with a I tab 4 with a central spring 6.

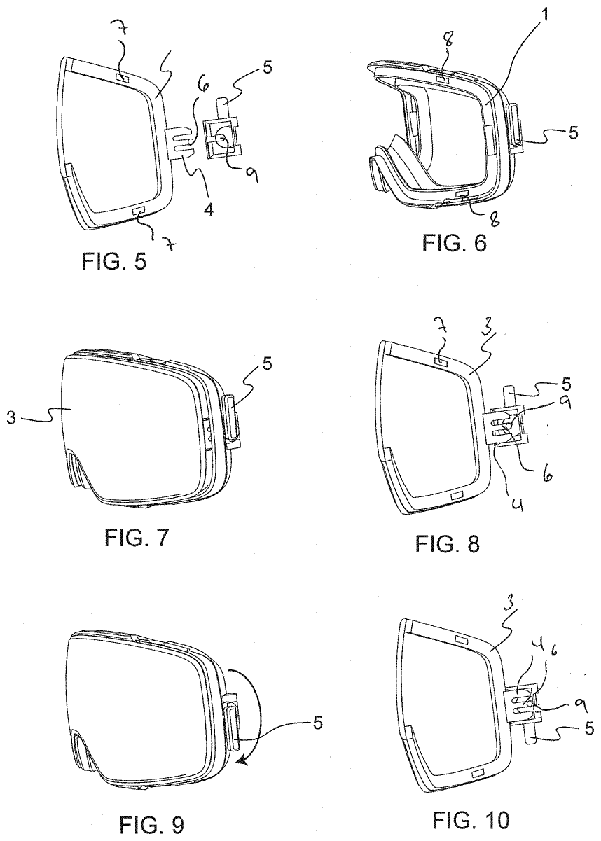

FIGS. 5 and 6 show the lens interchanging mechanism according to this invention.

The lens panel, made from injection molded nylon, contains magnets 7 for locating the lens in the correct location. These magnets are not for holding the lens. The lens panel is laminated between the outer lens and the inner lens.

The lens bedding 1, which is a part of the frame, contains magnets 8 for locating the lens, as well as a housing for the locking lever 5.

The tab 4 with central spring 6 is provided centrally on the lens panel. This central spring 6 of the tab 4 is to be received by the locking lever 5.

The locking lever 5 is provided with notch 9 to receive the central spring 6 of tab 4 and lock the lens panel in place by a half turn rotation of the lever 5.

The lens 3 is placed on the frame 1 and is automatically located correctly due to the magnets 7, 8 provided on the frame and the lens panel. FIG. 7 shows the frame with the lens thus fitted.

FIG. 8 is a cross section view of the lens and lens panel which shows how the central spring 6 of the tab 4 in the lens panel is automatically located in the notch 9 in the locking lever 5 but keeping a gap of say 5 mm from the frame.

As shown in FIGS. 9 and 10, when the locking lever is rotated, the lens is moved into the frame providing a secure attachment of the frame, as well as a draft-free fit.

When reversing the function to change lenses, the lever 5 is released and this allows the frame to present the lens protruding 5 mm from the frame. This in turn, allows the user to grab the edge of the lens without having to touch the lens surface, which is most likely to leave finger marks on the lens. This is a major advantage of the interchanging mechanism of the goggles according to the present invention.

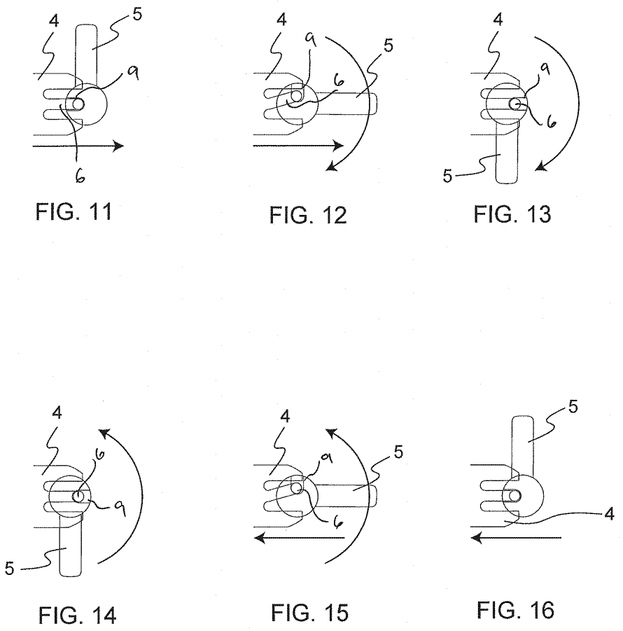

Reference is now made to FIGS. 11-16 which are detailed illustrations of the lens lock and unlock functions showing the tab attached to the lens panel and the rotating locking lever. These figures present a diagrammatic representation of the lens change concept according to the invention. It has two parts, i.e., locking the lens in place and releasing the lens.

To lock the lens in place:

As shown in FIG. 11, when the lens is inserted into the frame, it is protruding 5 mm from the frame. At this point, the central spring 6 of the tab 4 enters the notch 9 in the locking lever 5.

Next, the locking lever 5 is rotated downwards for locking the lens, as shown in FIG. 12. When rotating the locking lever 5, the central spring 6 of the tab 4 moves up. This is clearly shown in FIG. 12.

FIG. 13 shows how, when the locking lever 5 is fully rotated to its bottommost position, the lens moves 5 mm inwards. At this point, the central spring 6 in the tab 4 releases its tension and locks the lens in its place.

Thus, the lens is locked in place.

To Release the Lens:

As shown in FIG. 14, the locking lever is rotated upwards to release the lens. While the locking lever 5 is rotated upwards for releasing the lens, the center spring 6 in the tab 4 is pushed up. This is shown in FIG. 15.

FIG. 16 shows the position when the locking lever 5 is in the fully open position. At this point, the lens is unlocked and pushed out of the frame by 5 mm, so that the lens can easily be changed without touching the surface of the lens. This way, the risk of leaving finger marks on the lens surface is totally avoided.

The invention is particularly distinguished form the prior art with the locking device, which makes it possible for the user to place and remove the lens part without having to touch the surface of the lens, since the lenses are protruding out from the frame when the locking lever 5 is released. Another advantage is that it is easier for the user to grab the lens when he is wearing gloves or mittens.

The present invention has been described with reference to a preferred embodiment and some drawings for the sake of understanding only and it should be clear to persons skilled in the art that the present invention includes all legitimate modifications within the ambit of what has been hereinbefore and claimed in the appended claims.

1. A ski goggles with detachable lens comprising:
a frame constituted by a lens bedding part for receiving lens and being attached to a soft polymer frame part, so as to work as one piece, such that a lens panel is defined and laminated between the outer lens and the inner lens;
wherein magnets are provided in the lens bedding part and the lens panel for automatic and correct location of the lens; and
wherein a tab with central spring is provided on the lens panel, and a rotatable locking lever having a rotational axis, is provided on the lens bedding part, the locking lever is provided with a notch, where the bottom of the notch is offset from the rotational axis of the locking lever, where the central spring of the tab is received by a notch in the locking lever, where rotation of the locking lever in one direction pulls the lens towards the lens bedding part, the magnets are adapted to locate the lens on the lens bedding part such that once the locking lever is rotated in the opposite direction to release the engagement between the notch and central spring is released, the lens will protrude out of the lens bedding part keeping a gap between the lens and the lens bedding part.

2. The ski goggles with detachable lens according to claim 1, wherein the lens panel is made from injection molded nylon.

3. The ski goggles with detachable lens according to claim 1, wherein the lens bedding is made from carbon fibre reinforced nylon and the soft polymer frame part is made of thermoplastic polyurethane.

4. The ski goggles with detachable lens according to claim 1, wherein foam is provided towards the face of user and the foam is attached to the frame part.

5. A method of locking a lens in place in the ski goggles with detachable lens according to claim 1, the method comprising the steps of:
inserting the lens into the frame with the lens protruding out from the frame leaving a gap, when a central spring of a tab enters a notch in the locking lever;
rotating the locking lever downwards for locking the lens, when the central spring in the tab moves up; and
rotating the locking lever fully to its bottom most position, when the lens moves inwards, whereby the central spring in the tab releases its tension and locks the lens in its place.

6. A method of unlocking a lens which is in place in the ski goggles with detachable lens according to claim 1, the method comprising:
rotating the locking lever upwards to release the lens, when the central spring of the tab is pushed up; and
unlocking the lens and pushing it out of the frame to protrude out leaving a gap from the frame, when the locking lever is in the fully open position, whereby the lens can easily be changed without touching its surface.

7. The method according to claim 5 wherein, in the last step, the lens moves inwards by 5 mm.

8. The method according to claim 6 wherein, in the last step, the lens protrudes out of the frame leaving a gap of 5 mm.
